(19) United States
(12) Patent Application Publication
(10) Pub. No.: US 2010/0137704 A1
(43) Pub. Date: Jun. 3, 2010

(54) MEDICAL MATS WITH ELECTRICAL PATHS AND METHODS FOR USING THE SAME

(75) Inventors: Kamal Vij, Chandler, AZ (US); Kimble Jenkins, Memphis, TN (US)

Correspondence Address: MYERS BIGEL SIBLEY & SAJOVEC, PO BOX 37428, RALEIGH, NC, 27627, US

(73) Assignee: SurgiVision, Inc., Memphis, TN (US)

(21) Appl. No.: 12/627,587

(22) Filed: Nov. 30, 2009

Related U.S. Application Data
(60) Provisional application No. 61/119,149, filed on Dec. 2, 2008.

(51) Int. Cl. A61B 5/055; A61B 17/00; A61N 1/04; A61F 7/00; A61B 18/02; A61B 18/14

(52) U.S. Cl. 600/422; 606/1; 607/142; 607/112; 606/20; 606/41

(57) ABSTRACT

The disclosure describes medical mats that provide electrical paths with connectors that connect to various electronic medical or surgical tools. The medical mats can reduce the lengths of cables and define routes that preventing cross-over, looping and/or bunching of loose lengths of long cables.

### RELATED APPLICATIONS

This application claims the benefit of priority of and priority to U.S. Provisional Application Ser. No. 61/119,149, filed Dec. 2, 2008, the contents of which are hereby incorporated by reference as if recited in full herein.

### FIELD OF THE INVENTION

The present invention relates generally to medical devices used during medical procedures and may be particularly suitable for MRI-guided interventional procedures.

### BACKGROUND OF THE INVENTION

Some medical and surgical procedures use interventional or monitoring devices with relatively long lengths of various “loose” cables to connect to different electronic power and control systems during the procedures.

### SUMMARY OF EMBODIMENTS OF THE INVENTION

Embodiments of the invention may reduce lengths of loose cables and/or tubes conventionally used in medical procedures.

Some embodiments are directed to medical mats that include: (a) a mat body; (b) a plurality of discrete electrical paths in or on the mat body extending between at least one inlet location and a plurality of different outlet locations spaced apart about the mat body; (c) at least one inlet connector held by the mat body residing at the at least one inlet location in communication with one or more of the electrical paths; and (d) a plurality of outlet connectors held by the mat body at the different outlet locations, a respective outlet connector in communication with one or more of the electrical paths.

The mat body has a perimeter and the at least one inlet location may reside at a first perimeter edge portion and the plurality of different outlet locations may reside at different perimeter edge portions. In some embodiments, the inlet and outlet connectors can all reside on edge portions of the mat body. For example, at least one of the inlet and outlet connectors can reside on a primary upper surface of the mat body and other connectors can reside at outer edge portions of the mat body.

In some particular embodiments, the mat includes an electrical ground pad and/or external defibrillator pads. In some embodiments, the mat may optionally include at least one surface coil and/or at least one gradient receive coil.

Some embodiments are directed to systems. The systems include: (a) at least one medical mat having a plurality of discrete electrical paths that extend from at least one electrical input connector to a plurality of spaced apart electrical outlet connectors; (b) a first diagnostic or therapeutic medical tool with a first lead that connects to the at least one input connector and a second lead that connects to one of the outlet connectors; and (c) a second diagnostic or therapeutic medical tool with a first lead that connects to the at least one input connector and a second lead that connects to a different one of the outlet connectors.

The first tool can be an external ECG monitor and the second tool can be a cryogenic or electrode ablation catheter.

Still other embodiments are directed to methods of connecting medical instruments for a medical procedure. The methods can include: (a) placing at least one medical mat on a patient support surface, the at least one medical mat having defined electrical paths from at least one electrical input connector to spaced apart electrical output connectors; (b) having a patient lay on the at least one medical mat; (c) attaching at least one electrical input lead to the at least one input connector; and (d) connecting electrical output leads in communication with different medical tools to the output connectors.

A medical procedure can be carried out after the attaching and connecting steps using the medical tools. The placing step can be carried out by placing the at least one mat on a gantry of an MRI Scanner system.

Embodiments of the invention provide medical mats which define electrical paths that may reduce problems associated with conventional loose cables and/or tubes used during a medical procedure. The mats can help control the orientation and routing of electrical leads and/or cables used during a medical procedure to reduce “kinking” and/or electrical shorts from same, fluid in connectors and tangling of loose cables, and/or improve patient transportability.

Further features, advantages and details of the present invention will be appreciated by those of ordinary skill in the art from a reading of the figures and the detailed description of the preferred embodiments that follow, such description being merely illustrative of the present invention. Features described with respect with one embodiment can be incorporated with other embodiments although not specifically discussed therewith. That is, it is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. The foregoing and other aspects of the present invention are explained in detail in the specification set forth below.

### BRIEF DESCRIPTION OF THE DRAWINGS

FIG. 1A is a schematic top view of a medical mat according to embodiments of the present invention.

FIG. 1B is a schematic top view of a medical mat having “spare” electrical paths according to embodiments of the present invention.

FIG. 2 is a schematic top view of the medical mat shown in FIG. 1A according to embodiments of the present invention.

FIG. 3 is a schematic top view of a medical mat according to other embodiments of the present invention.

FIG. 4 is a schematic top view of a medical mat according to yet other embodiments of the present invention.

FIG. 5 is a schematic top view of a medical mat according to additional embodiments of the present invention.

FIG. 6 is a schematic top view of a medical mat according to some embodiments of the present invention.

FIG. 7 is a schematic illustration of a system using a medical mat according to embodiments of the present invention.

FIGS. 8A and 8B are digital photographs of known cardiac EP systems with cables that are used to connect various medical tools that may benefit from the use of a medical mat according to embodiments of the present invention.

FIGS. 9A-9D are side view schematic illustrations of medical mats that can be used in different manners according to embodiments of the present invention.

FIG. 10A is a schematic top view of a medical mat according to some embodiments of the present invention.

FIGS. 10B and 10C are end views of the mat shown in FIG. 10A with exemplary profiles according to embodiments of the present invention.

FIG. 11 is a sectional view of an exemplary mat according to embodiments of the present invention.

FIG. 12 is a sectional view of another exemplary mat according to embodiments of the present invention.

FIG. 13 is a sectional view of another exemplary mat according to yet other embodiments of the present invention.

FIG. 14 is a sectional view of yet another exemplary mat according to embodiments of the present invention.

FIG. 15A is an end or side view of another exemplary mat according to additional embodiments of the present invention.

FIG. 15B is a sectional view of an exemplary mat similar to that shown in FIG. 15A but with internal electrical paths as well as the external grooves according to embodiments of the present invention.

FIG. 15C is a top perspective view of the mat shown in FIG. 15A or 15B according to embodiments of the present invention.

FIG. 16 is a schematic perspective view of an MRI scanner using a medical mat according to embodiments of the present invention.

FIG. 17 is a schematic illustration of an exemplary electrical path with integral filters in the mats according to embodiments of the present invention.

FIG. 18 is a flow chart of exemplary steps that can be used to carry out embodiments of the present invention

### DETAILED DESCRIPTION OF EMBODIMENTS OF THE INVENTION

The present invention now is described more fully hereinafter with reference to the accompanying drawings, in which some embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

Like numbers refer to like elements throughout. In the figures, the thickness of certain lines, layers, components, elements or features may be exaggerated for clarity.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus, the exemplary term “under” can encompass both an orientation of “over” and “under”. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

The term “mat” refers to a substantially flat device such as a pad, cover or other device with electrical paths extending through portions thereof and that is typically resilient or flexible but has sufficient rigidity and/or thickness to hold the electrical paths and/or leads in a manner that does not cause discomfort to a user or patient. The mat can comprise an elastomeric and/or fabric body. The electrical paths held by the mat can be formed as internal wires, filars, metallic traces or cables. For MRI procedures, the conductive material used to form the electrical paths in the mat (e.g., the leads held by the mat) can be non-ferromagnetic. The term “lead” means an electrical path created by one or more wires. The wires are typically insulated wires, particularly where exposed. The term “cable” is used interchangeably with the term lead.

The term “MRI-compatible” means that a device is safe for use in an MRI environment and/or that a device that can operate as intended in an MRI environment and not introduce artifacts into MRI signal data. As such, if residing within the high-field strength region of the magnetic field of an MRI suite, the MRI-compatible device is typically made of a non-ferromagnetic MRI-compatible material(s) suitable to reside and/or operate in a high magnetic field environment. The term “high magnetic field” refers to magnetic fields above 0.5 T, typically between 1.5 T to 10 T. Although described as particularly suitable for MRI procedures or suites, the medical mats can also be used with CT or other imaging modalities, such as for example, for CT-guided cardiac EP procedures or robotic surgical procedures.

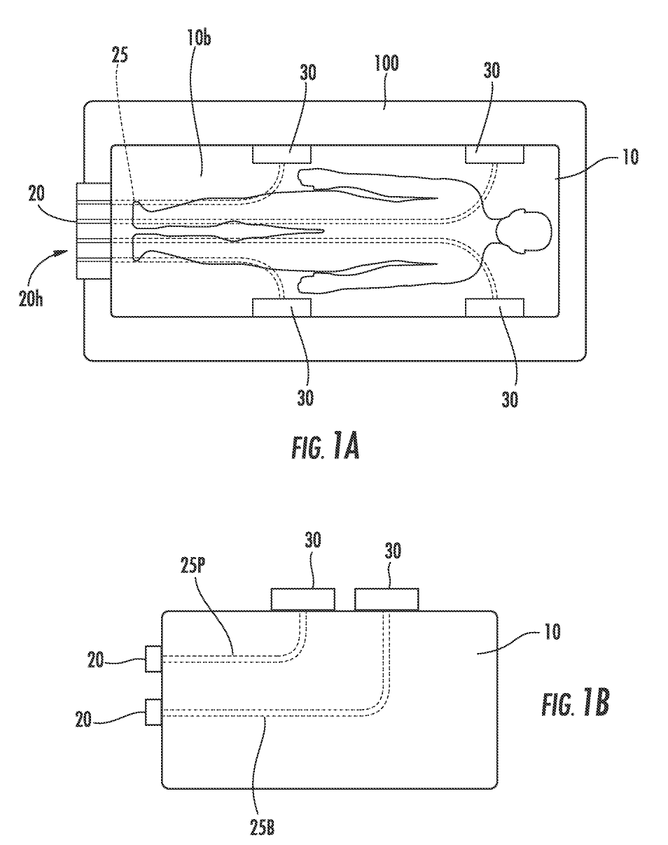

Referring now to the figures, FIG. 1A illustrates a medical mat 10 residing on a patient support surface 100. As shown, the mat 10 includes at least one electrical input connector 20 and a plurality of spaced apart electrical output connectors 30 with electrical paths 25 extending therebetween. The mat 10 can be configured to provide a desired number of discrete electrical paths 25, typically between about 5-1000, and more typically between about 10-500, such as between about 12-200, and, in some particular embodiments between about 60-120, but lesser or greater numbers of paths can be used. The mats 10 can be used with medical procedures for any subject, but are particularly suitable for animal and/or human subjects. The support surface 100 can be any support surface, typically associated with a hospital bed, a stretcher, an examination table or chair, a surgical table, a gantry of an imaging system such as a CT scanner or MRI scanner, and the like.

The medical mats 10 contemplated by embodiments of the present invention may be used with respect to any medical or surgical procedure including diagnostic, interventional or exploratory procedures. For example, the mats 10 can be used with ultrasound, X-ray, CT or other imaging modalities. The mats 10 may be particularly suitable for use with MRI-guided surgical procedures, such as cardiac procedures, including but not limited to cardiac EP procedures where heat or cryogenic ablation is used and where the procedure is carried out in an MRI scanner or MRI interventional suite.

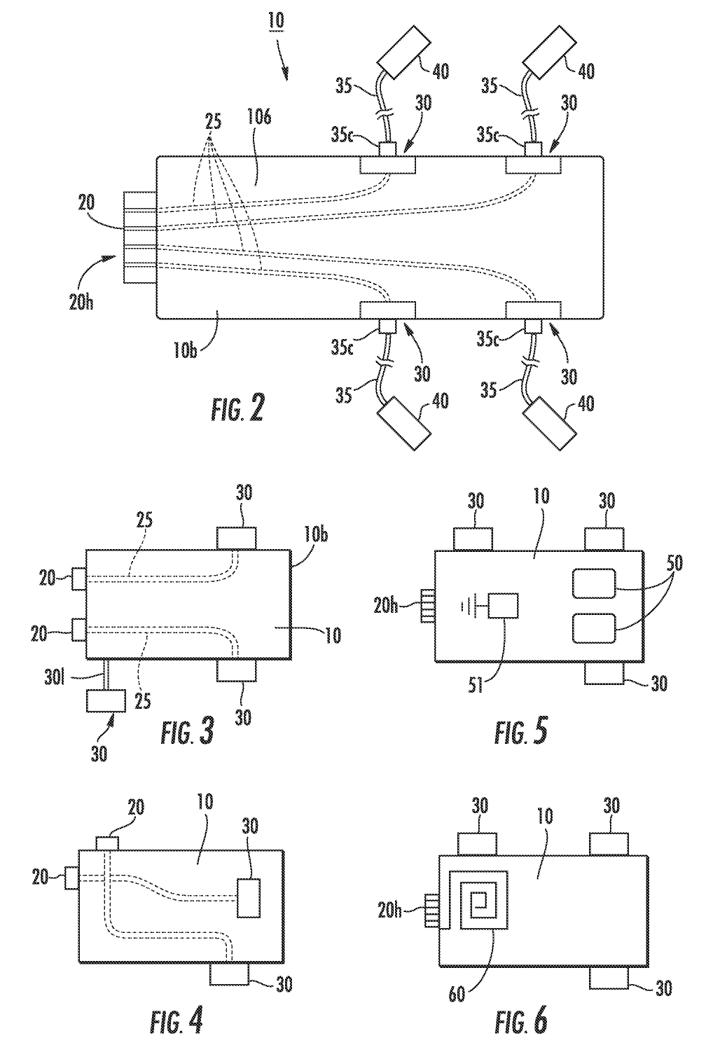

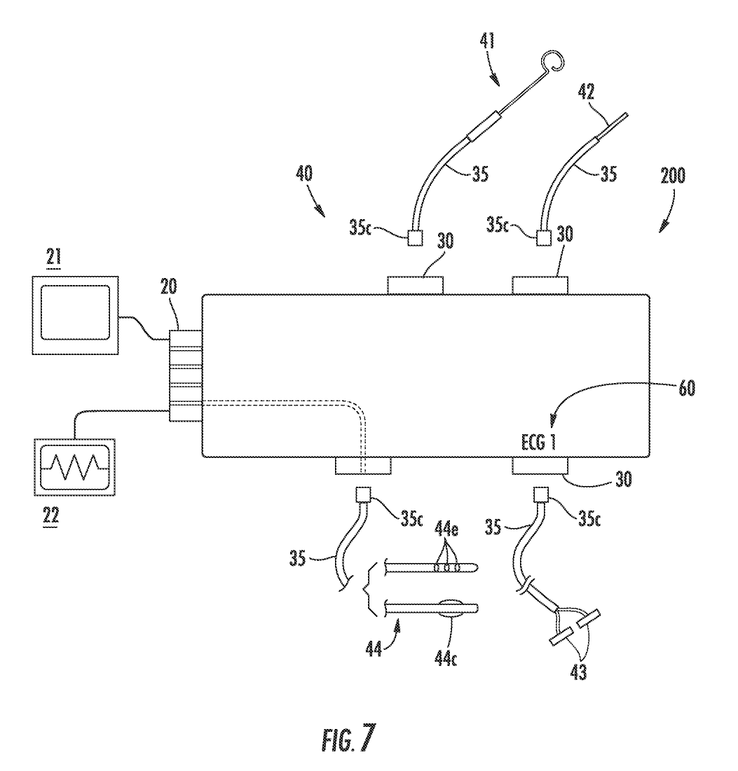

One or more of the electrical paths 25 can extend from a primary “input” end to branch out to other edge portions, e.g., side or end perimeter portions of the mat 10. As shown, for example, in FIGS. 1A and 2, in some embodiments, at least one electrical path 25 extends from the end (foot end) to a side of the patient support surface, and, as shown, the end is the electrical input “hub” 20h. The term “hub” means that a majority, typically all or substantially all, of the electrical paths start from one or more connectors/inputs at a “central” or hub location at an end or side of the mat. However, one or more (or all) of the electrical paths 25 may start and/or extend in different directions, e.g., across the support surface, from a side to an end or from end to end as well. FIG. 4 illustrates a side input connector 20 extending to an opposing and offset side location of the respective output connector 30. FIG. 3 illustrates two spaced apart input connectors 20 on one end of the mat 10 branching to a respective output connector 30 on opposing sides of the mat.

As shown in FIG. 1B, the mat 10 can include “spare” or back-up electrical paths 25b (e.g., wires) with connectors 20, 30 for one or more of the inlet and outlet locations, different connector types, and/or target medical instruments on one or more of the electrical paths 25 indicated that can be used in the event of failure of the primary electrical path 25p. As shown in FIG. 1A, the mat 10 has a mat body 10h and may reside under the patient. The mat can be configured to allow a patient to lie on the mat 10 during the medical procedure.

The mats 10 can be used to control the orientation and/or reduce the lengths of loose cables associated with conventional procedures (see, e.g., FIGS. 8A, 8B) for ease in access to the patient or tools, to improve patient transportability, to provide a more efficient medical set-up, and/or to arrange the cables so that they do not loop or cross-over each other to inhibit heating or burns that may be induced due to the RF environment in MRI-guided procedures.

The mat 10 can be sterile (meaning that it meets clinical cleanliness standards for medical procedures) and may optionally be single-use disposable. Alternatively, or additionally, a sterile cover or case can be used as appropriate. The mat 10 can be a “universal” mat configured for multiple different procedures or may be procedure-specific, e.g., a cardiac mat and/or a cardiac EP (electrophysiology) mat.

The mat 10 may directly or indirectly contact the patient. The mat 10 can be releasably attached to the support surface 100 using adhesives, straps or the like. The mat 10 may be configured to be used without such an affirmative attachment feature, and may optionally include an anti-slide or textured surface or a contact surface with an increased friction material or coating that inhibits sliding.

The mat 10 may cover all or substantially all of the patient support surface 100 as shown in FIG. 1, or the mat 10 may be smaller to occupy only a sub-portion of the support surface, such as between about 50-75% or 25-50% of the surface area of the support surface 100. Typically, the mat 10 is configured to extend from one end portion of the support surface with a width that is typically between about 50-110% of that of the support surface 100 to be able to route electrical paths to a desired location that limits the length of the external leads that connect to a medical tool or instrument. In some embodiments, the mat 10 may extend over the edges of the support surface 100 or reside inside the bounds of the support surface 100.

The electrical connectors 20 or 30 can be standard connectors such as BNC connectors, coaxial connectors and the like or the connectors 20, 30 may be customized connectors. The input connector(s) 20 can be provided as a main cable connection hub 20h of closely spaced electrical connectors. As shown in FIG. 1A, the hub 20h can reside on an outer edge portion of the mat on one side (shown as an end or short side). For some particular embodiments, such as cardiac EP procedures, the outlet connectors typically include Hypertronoics™ multi-pin connectors for an ablation catheter. ECG leads, coronary sinus catheter, lasso catheter, defibrillation and pacing devices.

Some or all of the connectors 30 and 20 can reside within the bounds of the mat body 10b as shown in FIG. 2. The electrical paths 25 can be configured accordingly to terminate at outer perimeter edges. Alternatively, as shown in FIG. 4, one or more of the connectors 20, 30 can extend from or reside on a primary upper or lower surface in a medial or inset length from the edge or end of the mat 10. As shown in FIG. 3, one or more of the connectors 20, 30 may extend outward a distance from the mat body 10b to facilitate ease of connection. While some of the connectors 20, 30 are shown as residing adjacent the bounds of the mat body, FIG. 3 also illustrates that one or more of the connectors 20, 30 (shown as an output connector 30) can reside a further distance with a length of lead 301 extending out of the mat body 10b. Typically, the length of the outwardly extending lead 301 is typically relatively short to inhibit contact with the floor and/or kinking or cross-over with other leads and/or facilitate patient transport with the mat in place, at least where the mat 10 is used on the support surface or on the patient (e.g., between about 1 inch to about 4 feet, typically between about 6 inches to 2 feet). The mat 10 may be configured with most connectors adjacent the mat body perimeter or primary surface, with a limited number of longer leads 301, such as a single one of the longer leads 301 or a single one of this type lead 301 on each side or end of the mat 10 to allow longer lengths for accommodating different procedures. In some embodiments, the connectors 20, 30 all reside adjacent the mat body 10b (accessible at perimeter edges and/or via a primary surface). Interface extension leads (e.g., cables) can be provided to allow custom set-up of different medical procedures where extra lengths may be desired (not shown).

FIG. 2 also illustrates that the mat 10 can connect several medical tools 40 using the outlet connectors 30, the electrical paths 25 and the input connector(s) 20. That is, the tools 40 include external leads 35 with connectors 35c that connect to the outlet connectors 30 to connect to the power and/or input systems via one or more input connectors 20. Because the tools 40 connect to the mat 10 rather than all the way back to their respective system hardware, the leads 35 can be shorter than conventional leads, typically between 1-5 feet, eliminating or reducing the mass of loose cables used in the past (see, e.g., FIGS. 8A, 8B).

The different systems can be positioned at one common end of the patient support surface or placed where suitable. As will be discussed further below, one of the tools 40 can be an ECG monitor and the monitor can be at the input end and connect through the input connector 20 with one or more cardiac cycle sensors (e.g., 10 or more electrodes) connecting through a connector 35e to a respective connector 30 and path 25 to provide the output to the monitor. Each sensor may connect to separate connectors 30 or grouped to connect otherwise.

The connectors 30 may be color-coded to the external lead to Facilitate set-up and proper connection. The connectors 30 may have different shapes to inhibit improper connection of external leads. As shown in FIG. 7, the mat 10 can include visual indicia 60 (shown as “ECG1”) that indicates which external lead 35 (FIG. 2) connects to which connector 30. Although the indicia 60 is shown in FIG. 7 with respect to an outlet connector 30, the mat 10 can also include visual indicia for inlet connections as well.

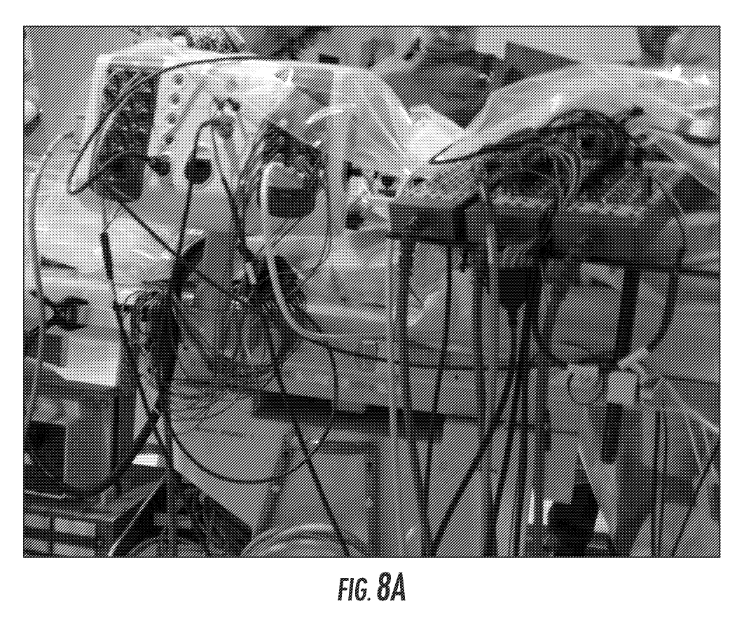

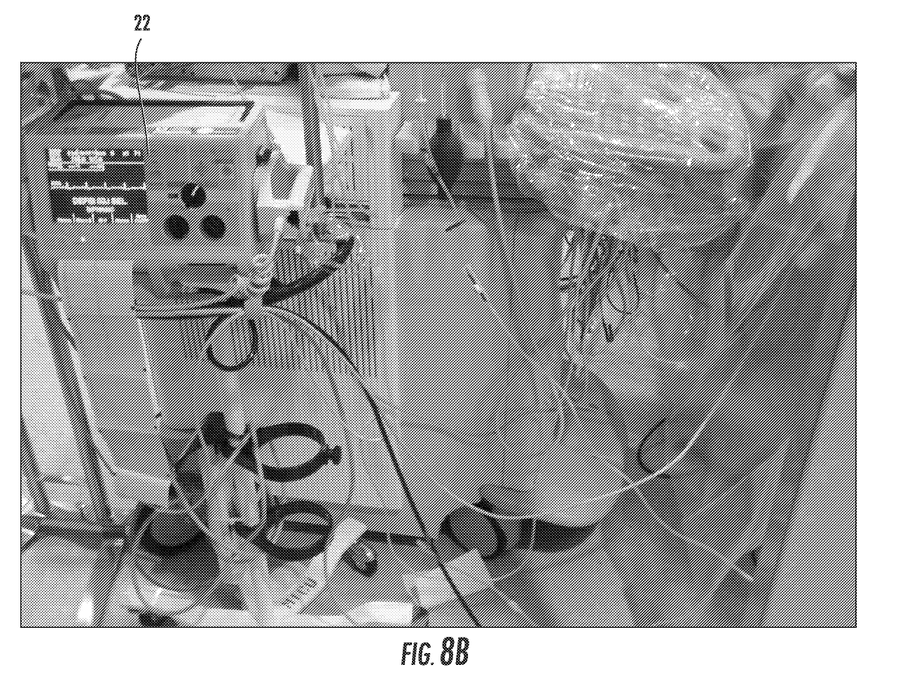

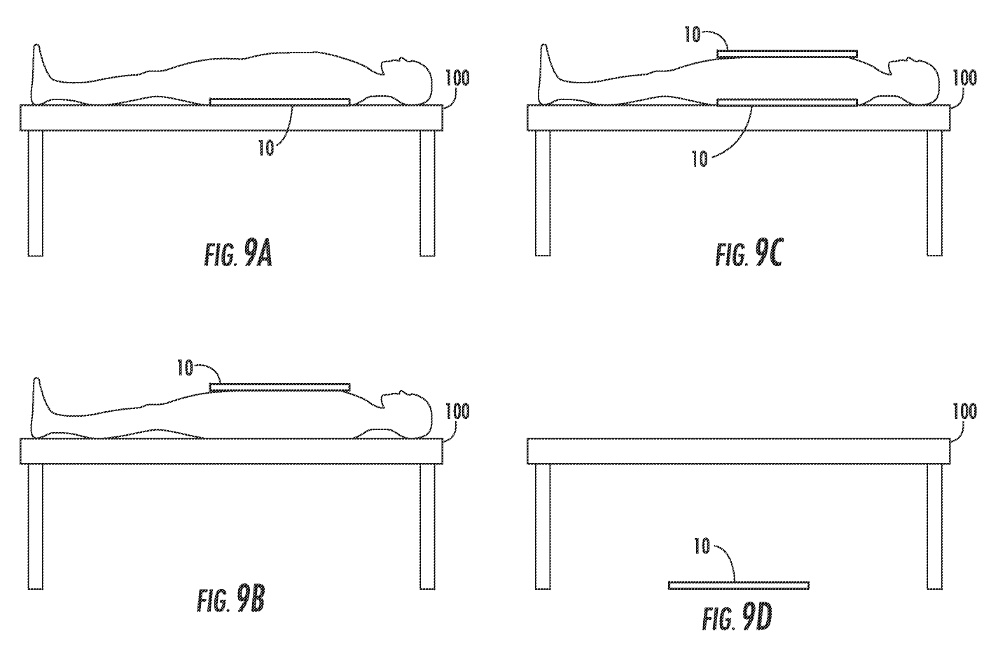

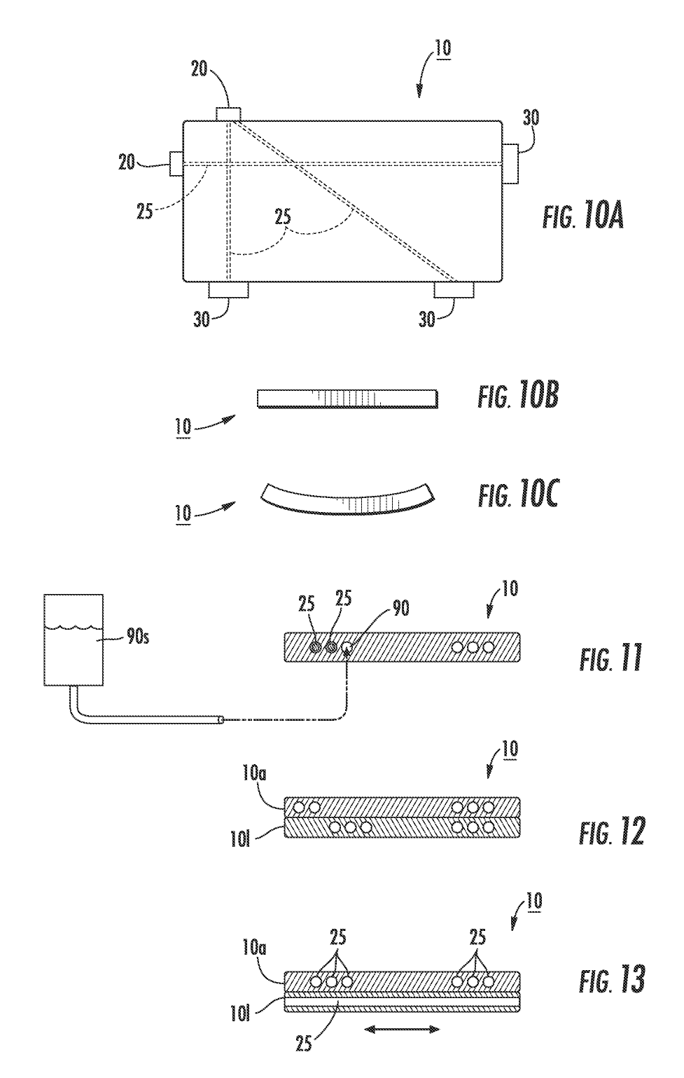

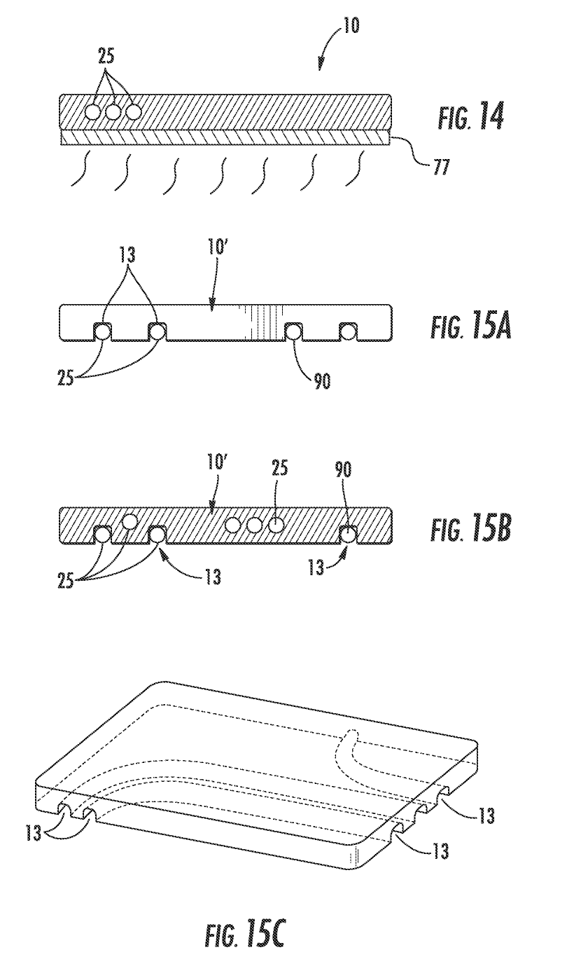

FIGS. 5 and 6 illustrate that the mat 10 can include integral components that may be useful for a medical procedure. FIG. 5 illustrates that the mat 50 can include defibrillation pads 50p and/or a grounding pad 51. FIG. 6 illustrates a mat that may be particularly useful for MRI procedures, with the mat 10 including one or more coils 60, such as one or more gradient coils and/or one or more surface coils (the surface coil can be configured to transmit and/or receive MRI signals). The coil(s) 60 can connect to the MR scanner using a connector on the mat 10.

FIG. 7 illustrates an exemplary system 200 with the mat 10 and four different tools 40 connected by the mat to their sub-systems for an MRI-guided cardiac EP procedure. As shown, the system 200 includes a lasso catheter 41, a sinus catheter 42, ECG sensors 43, and an ablation catheter 44 e.g., heat-based (such as a single or multi-electrode 44e ablation catheter) or cryogenic-based (such as a cryo-ablative probe 44c); each can have a connector 35c and lead 35 that connects to the mat 10 for the electrical path 25 to the desired input connection 20. The sub-systems include a clinician workstation 21 with display/monitor for display of EP mapping and a display for the ECG Monitor 22. In operation, ablation energy is applied to target cardiac tissue using the ablation catheter 44, the ECG signal can be monitored using the internal ECG signals and/or using external leads 35 of the ECG sensors 43, and the EP of the heart is mapped (and displayed) (generating an electroanatomical map) using the lasso and/or sinus catheters. Other patient monitoring leads/systems can be used including respiratory and blood pressure, for example.

EP clinical procedures involve recording and displaying in real-time numerous external and internal ECG signals. A coronary sinus catheter simultaneously measures as many as fourteen different ECG signals from inside the patient's heart (and may measure less or more than fourteen). In such an embodiment, these signals come down fourteen different wires down the catheter and the output connector has at least fourteen pins to transfer these measurements to the subsystems that process these measurements. Similarly, the output connector of the lasso catheter has eight ECG signal pins while the ablation catheter has between about two to four ECG signal pins and two tip temperature pins. The external ECG can be recorded separately, typically using twelve (12) patch electrodes. Besides recording ECG, some of the same connector pins are used for externally pacing and/or defibrillating the patient's heart. In the ECG monitor subsystem fractionated ECG signals are displayed on a monitor and these signals guide the physician to specific target sites. A physician refers back to this monitor to confirm success of ablation procedure. Further, these ECG signals are one of the inputs used to generate the electro-anatomical map by the workstation subsystem. FIGS. 8A and 8B show the number of loose cables used with conventional systems that can be improved using the mats contemplated by embodiments of the present invention. FIG. 8A and FIG. 8B show the back of the patient interface unit in a typical electro-physiology procedure room. There are a number of cables hanging loose. They can be managed better and located consistently by using one or more of the mats contemplated by embodiments of the invention.

FIG. 9A illustrates that the mat 10 can reside under the patient on the support surface 100. FIG. 9B illustrates that the mat 10 can reside over the patient. FIG. 9C illustrates that two mats 10 can be used, one above the patient and one below (two side-by-side mats may also be used). FIG. 9D illustrates that the mat 10 may optionally be placed on the floor or a shelf under the patient support surface and is not required to contact the patient. The latter may require longer lengths of cables that may not be desirable for some uses.

The mat 10 can be formed of one or more materials. The outer surfaces can be formed of a material(s) that is substantially impermeable to fluids. According to some embodiments, the mat 10 can include a biocompatible polymeric material, such as those suitable for use in MRI systems. Exemplary polymeric materials may include polyvinyl, PET, silicone, polyethylene, polyurethane, and/or polyamide. Where the mat 10 contacts the patient, the mat 10 may be configured to provide heating or cooling as desired for patient comfort or treatment. Where the patient lies on the mat 10, the mat 10 may be configured to provide cushioning using an air pocket, flexible soft material such as memory foam and/or gel material for patient comfort. Where the mat lies on the patient, the mat can be configured to be light weight and substantially conformable to the patient.

FIG. 10A illustrates that the mat 10 can be configured with input connectors 20 on two different sides of the bed (on one short side and on one long side) and that one input connector 20 can communicate with a plurality of output connectors 30 and electrical paths 25. FIG. 10A also illustrates the mat 10 with a perimeter shape that is substantially rectangular. However, the mat 10 can be provided in other shapes, including, for example, square, round, oblong and other geometric shapes. FIGS. 10B and 10C show two different end view profiles, one which may taper up to “cradle” the patient and the other is substantially planar. Again, other shapes may be used (such as ramped shapes and the like).

FIG. 11 illustrates that the electrical paths 25 can be encased by the body of the mat 10. One or more of the electrical paths 25 can be formed using one or groups of wires. As also shown by FIG. 11, the mat 10 can also include one or more fluid passages/tubes 90 that can be used to connect to a fluid source 90s to allow medicines and/or drugs to be delivered to a patient, such as via IV drips, or that circulates fluid for heating and/or cooling.

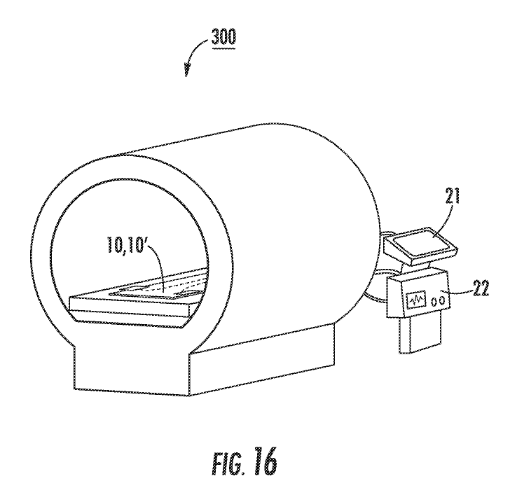

FIGS. 12 and 13 show that the mat 10 can be a multi-layered mat with electrical paths 25 provided in each layer (shown as two layers 10u, 10l but more layers can be used). One or more fluid passages 90 (FIG. 11) can be provided in one or more of the layers. The fluid passages 90 can be for gas or liquid. The fluid passages 90 can be formed integral to the mat as fluid-tight channels or may be provided as tubular or other fluid-tight conduits held in one or more of the passages of the mat 10. FIG. 12 also shows the electrical paths 25 can reside laterally spaced apart for a portion of their length in each layer 10u, 10l but can be oriented to run substantially the same direction for a portion or substantially all of their length. FIG. 13 shows that one layer can run electrical paths in one direction and the other layer can run an electrical path orthogonal to the direction of the other layer. As shown, the bottom layer 10l runs the electrical path(s) 25 laterally while the upper layer 10u runs the electrical paths 25 longitudinally.

FIG. 14 shows that the mat 10 can be configured to heat and/or cool the patient. As noted above, the mat 10 can circulate fluid for heating and/or cooling, such as by using channels formed therein. Alternatively, other heating and cooling mechanisms or materials may be used as is known to those of skill in the art. For example, a microwave-activated material can be used to provide the heating or a heating coil, resistive element or electrode array may be used. For MRI use, the length of the leads in the mat 10 can create heating based on current induced heating. Alternatively, the leads can be configured with RF chokes, Balun circuits, high impedance, and/or a series of reverse and forwards sections. See, e.g., U.S. Patent Application Publication No. US-2008-0243218-A; and U.S. patent application Ser. No. 112/090,583, the contents of which are hereby incorporated by reference as if recited in full herein. The microwave-activated material can be done provided a layer that can be placed on/attached to the mat prior to use. Similarly, a coolant cushion or layer can be placed on the mat prior to use.

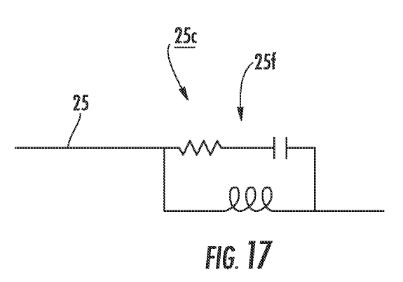

FIG. 15A illustrates a mat 10′ with external grooves 13 that can snugly hold a lead, a wire or groups of wire that form the electrical paths 25 and/or a conduit for providing a fluid path 90. FIG. 15B shows the mat 10′ can also include internal electrical paths 25 as well as the electrical and/or fluid paths held by the external grooves 13. FIG. 15C shows an example of the mat 10′ which routes one or more conduits, cables, wires or groups of wires or cables to different locations along the perimeter of the mat 10′.

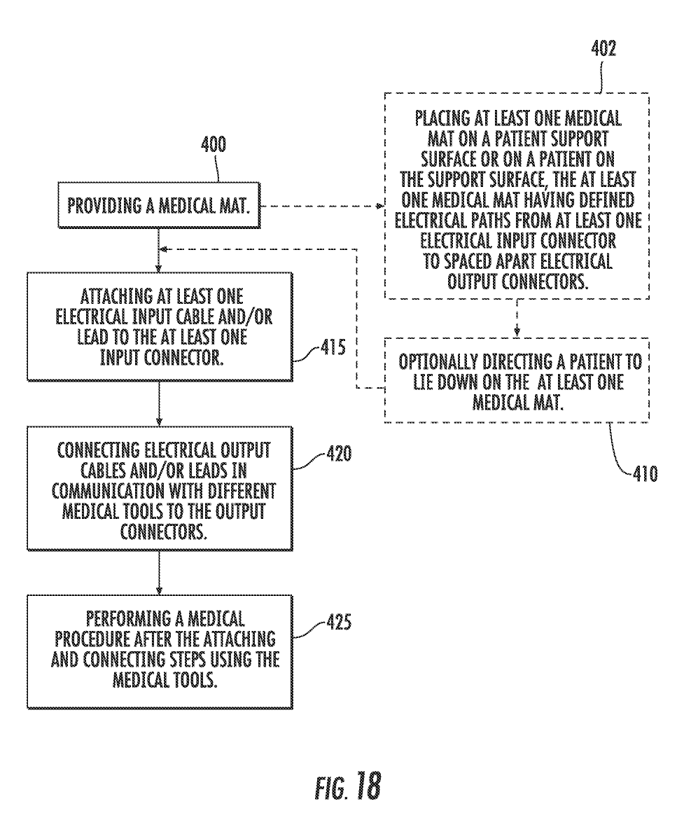

FIG. 16 illustrates the mat 10, 10′ on a gantry of an MRI scanner 300 connected to various subsystems 21, 22, such as, for example, those used for cardiac EP procedures. The mat 10, 10′ can be used for other MRI procedures including brain surgery and other cardiac surgeries, and interventional procedures for other anatomical locations of the body.

FIG. 17 illustrates that one or more of the electrical paths 25 in the mat 10, 10′ can include circuits 25c such as filters that can facilitate signal acquisition or transmission (e.g., reduce noise, improve SNR and the like).

FIG. 18 is a block diagram of exemplary steps that can be used to carry out some embodiments of the present invention, such as connecting medical instruments for a medical procedure. A medical mat is provided (block 400). The medical mat having defined electrical paths from at least one electrical input connector to spaced apart electrical output connectors. At least one electrical input lead can be attached to the at least one input connector (block 415). Electrical output leads in communication with different medical tools can be connected to the output connectors (block 420). A medical procedure can be performed after the attaching and connecting steps using the medical tools (block 425). Optionally, the mat can be placed on a gantry of an MRI Scanner system. Optionally, at least one medical mat can be placed on a patient support surface before a patient resides on a gantry or support surface or the mat can be placed over the patient after the patient is on the support surface or gantry (block 402). Also optionally, the patient can be directed to lay (or can be placed) on the at least one medical mat on the support surface (block 410).

The mats 10 can be fabricated in any suitable manner. In some embodiments, it is contemplated that the mat can be molded and the electrical paths can be molded internal to the exterior surface of the mat. In some embodiments, a first layer of the mat can be molded with mold forms defining open cavities or channels that will hold the wires in the desired locations and orientations and route them to the outlet locations. The mold forms can be removed and wires placed into those cavities or channels, and a second layer can be molded over the wires (or tubes for fluid channels where used) to encase them and hold them in position (when fabricating the internal wire path configuration). Alternatively, the mat can be molded in one step with the mold form channels left in position and the wires threaded through the channels (either before or after the molding step). However, the mat is not required to be molded. In some alternate embodiments, fabric can be used to form the mat and the mat electrical paths can be formed by threading cables or wires through sewn or adhesively formed channels. The mats 10 can also be fabricated in yet other manners. The mats 10 and electrical paths 25 can be sterilized and packaged in a sterile package for medical use during a medical procedure.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the teachings and advantages of this invention. Accordingly, all such modifications are intended to be included within the scope of this invention as defined in the claims. The invention is defined by the following claims, with equivalents of the claims to be included therein.

That which is claimed is:

1. A medical mat comprising:
a mat body;
a plurality of discrete electrical paths in or on the mat body extending between at least one inlet location and a plurality of different outlet locations spaced apart about the mat body;
at least one inlet connector held by the mat body residing at the at least one inlet location in communication with one or more of the electrical paths; and
a plurality of outlet connectors held by the mat body at the different outlet locations, a respective outlet connector in communication with one or more of the electrical paths.

2. A medical mat according to claim 1, wherein the mat body has a perimeter, and wherein the at least one inlet location resides at a first perimeter edge portion and the plurality of different outlet locations reside at different perimeter edge portions.

3. A medical mat according to claim 1, wherein the inlet and outlet connectors all reside on edge portions of the mat body.

4. A medical mat according to claim 3, wherein at least one of the inlet and outlet connectors reside on a primary upper surface of the mat body and other connectors reside at outer edge portions of the mat body.

5. A medical mat according to claim 2, wherein the mat body is rectangular with pairs of opposing short and long sides, and the at least one inlet location resides at an edge portion of a short side and at least some of the outlet locations reside at edge portions of a long side.

6. A medical mat according to claim 1, further comprising a main cable connection hub as the at least one electrical input connector residing at a first perimeter side of the mat body in communication with the electrical paths.

7. A medical mat according to claim 1, wherein at least some of the electrical paths are defined by insulated wires that are encased by the mat body.

8. A medical mat according to claim 1, wherein, in use, the mat resides under a patient on a patient support surface.

9. A medical mat according to claim 1, wherein, in use, the mat resides above and on the patient.

10. A medical mat according to claim 1, wherein one of the outlet connectors is adapted to communicate with an ECG lead.

11. A medical mat according to claim 1, wherein the mat body comprises at least two layers of electrical paths.

12. A medical mat according to claim 1, wherein the mat comprises an electrical ground pad and/or external defibrillator pads.

13. A medical mat according to claim 1, further comprising at least one surface coil and/or at least one gradient receive coil.

14. A medical mat according to claim 1, wherein the mat body comprises a cushioning material.

15. A medical mat according to claim 1, wherein the mat body is configured to provide heating and/or cooling for patient comfort.

16. A medical mat according to claim 1, wherein the electrical paths are provided by between about 10-200 wires in the mat body.

17. A medical mat according to claim 1, wherein at least some of the outlet connectors are BNC connectors.

18. A medical mat according to claim 1, wherein the mat is a cardiac EP mat and the outlet connectors comprise a lasso catheter connector, a sinus catheter connector, an external ECG lead connector, and an ablation catheter connector.

19. A medical mat according to claim 1, wherein the mat is an MRI compatible mat adapted for MRI-guided surgical procedures.

20. A medical mat according to claim 1, further comprising at least one fluid channel that extends through the mat and is adapted to connect to a fluid source.

21. A system comprising:
at least one medical mat having a plurality of discrete electrical paths that extend from at least one electrical input connector to a plurality of spaced apart electrical outlet connectors;
a first diagnostic or therapeutic medical tool with a first lead that connects to the at least one input connector and a second lead that connects to one of the outlet connectors; and
a second diagnostic or therapeutic medical tool with a first lead that connects to the at least one input connector and a second lead that connects to a different one of the outlet connectors.

22. A system according to claim 21, wherein the first tool is an ECU monitor, and wherein the second tool is a cryogenic or electrode ablation catheter.

23. A system according to claim 21, wherein a patient lays on the at least one mat during use.

24. A system according to claim 21, wherein the at least one mat lies over a patient during use.

25. A system according to claim 21, wherein the at least one mat includes a first mat that resides under a patient and a second mat that resides over the patient.

26. A system according to claim 21, wherein the at least one mat also comprises a fluid channel for delivering a fluid to a patient during a medical procedure.

27. A mat for arranging medical leads in a fixed path, comprising:
a mat body with upper and lower primary surfaces, one of the surfaces comprising grooves, a respective groove configured to snugly engage a respective lead from one side of the mat body to another side of the body.

28. A mat according to claim 27, further comprising a plurality of electrical paths encased by the mat body and extending between inlet and outlet connectors.

29. A method of connecting medical instruments in preparation for a medical procedure, comprising:
providing at least one medical mat, the at least one medical mat having defined electrical paths from at least one electrical input connector to spaced apart electrical output connectors;
attaching at least one electrical input lead to the at least one input connector; and
connecting electrical output leads in communication with different medical tools to the output connectors thereby electrically connecting tools to electrical inputs for a medical procedure.

30. A method according to claim 29, wherein the providing step further comprises placing at least one medical mat on a gantry of an MRI Scanner system or over a patient on the gantry.

31. A method according to claim 29, further comprising performing a medical procedure after the attaching and connecting steps using the medical tools.
